(19) United States
(12) Patent Application Publication
(10) Pub. No.: US 2020/0151814 A1
(43) Pub. Date: May 14, 2020

(54) Methods and Systems for Scoring Healthcare Debt Obligations

(71) Applicant: John Michael Agnew, Columbus, GA (US)

(72) Inventor: John Michael Agnew, Columbus, GA (US)

(21) Appl. No.: 16/559,493

(22) Filed: Sep. 3, 2019

Related U.S. Application Data
(60) Provisional application No. 62/726,386, filed on Sep. 3, 2018.

(51) Int. Cl. G06Q 40/02; G16H 10/60; G06Q 10/10

(52) U.S. Cl. CPC G06Q 40/025; G06Q 10/10; G16H 10/60

(57) ABSTRACT

Systems and methods for a risk-based scoring to predict the collectability of a medical lien based on collection factors are disclose here. This allows the medical lien or debt to risk assessment. The modeling of risk includes obtaining accident/incident data, modeling and/or processing the health care data, and creating an output.

### TECHNICAL FIELD

This application relates to methods and systems for assessing the potential recovery of a healthcare lien for uninsured patients and for the patient portion of healthcare debts that are covered by managed healthcare plans through the identification of potential funding sources and the collection of data required by such potential funding sources.

### BACKGROUND

Various methods are known in the art for predicting the payment behavior of various categories of debtors in categories other than health care. Examples of such methods are disclosed in US Patent Application Nos.: US 2003/0212618 A1; US 2005/0197954 A1; US 2006/0287947 A1; US 2007/0208640 A1 and US 2007/0219885 A1, all hereby incorporated by reference. Because of different factors facing the healthcare industry, known payment behavior modeling techniques are generally not applicable to the healthcare industry. For example, healthcare providers, such as a hospital, or other acute or emergency care facility, are required by law to provide certain medical services irrespective of a patient's ability to pay under certain conditions. As such, statistical methods for payment of debtors other than healthcare providers are generally not applicable.

One of the causes of lengthy patient balance time periods is complex claims involving patients whose medical treatment is or may be the result of a motor vehicle accident. In such cases there are intricate processes required to successfully navigate the coordination of benefits process required to resolve balances for insured patients.

For balances related to uninsured patients there are many states which allow the provider to file a lien to allow for the collection of medical expenses directly from the proceeds of any personal injury claim brought by or on behalf of that patient related to the motor vehicle accident. Even if the cases are properly identified and the correct steps are taken to protect the hospitals interest and ability to recover their balances it is very difficult for the provider to have any understanding of the quality, value, likely duration, or complexity of the claim and it can be nearly impossible to accurately monitor these claims as a single category or based on age of the patient balance. There is an extreme lack of knowledge as to appropriate expectations on these account balances. Data required to allow for such analysis is not even available to the medical provider. Additional data specific to these claims must be gathered from the patient and from third parties and then correctly interpreted and used in the process. As a result of the nature of the lien process being dependent on the resolution of the underlying claim, it is very difficult to predict when payment should resolve. This makes the revenue cycle time period irregular. It also makes it difficult to monitor and correct unnecessary delays and delinquencies because they are not easily identified.

These issues cause the category of Motor Vehicle Accident related medical balances to be a disproportionately large problem for medical providers when compared to the management of other medical billing and collections scenarios.

Other attempts to address these issues have been inadequate because they are inefficient and irregular in data gathering. Most of the data gathering is in the form of unstructured data which must be manually manipulated. There is a lack of understanding of the value and quality and lack of ability to track the underlying personal injury claim. The only method available to manage these claims is to bundle them by age which can often be a completely irrelevant indicator of value. There is not a good method available for understanding the risk of nonpayment of any specific patient balance in this scenario.

### SUMMARY

Specific embodiments relate to systems and methods for a risk-based scoring system to predict the collectability of a medical lien based on collection factors. This allows the medical lien or debt to risk assess, and ultimately collected or discharged by the health care provider. In one embodiment, modeling risk includes obtaining accident/incident data, modeling and/or processing the health care data, and creating an output. The output may then be used to make business decisions. The variety of data (e.g., consumer data) in conjunction with several modeling/processing procedures to assess risk of recovering or not recovering a lien. Once the data is obtained by the evaluator, the rules engine can be programmed to create a set of questions structured to gather all of the required data for creating or modeling the data.

One embodiment includes a method for assessing debt obligations on an individual basis, having obtaining over a communication network connection, metadata corresponding to one or more potential health care debts from sources to obtain repayment information about each of the one or more debts; transforming the data into a profile for each debt which corresponds to an individual's obligation; and scoring the profile for predicted repayment of the debts, wherein the score correlates with the collectability of the debt. In one example the scoring includes transaction data based on a preexisting model to form a score for said credit account.

In another example, the method or system can include transmitting the score to a third party different from provider if said score reflects a low or high level of financial risk.

In another embodiment, the method including the steps of obtaining over a communication network connection, metadata corresponding to one or more potential health care debts from sources to obtain repayment information about each of the one or more debts; and coring with an algorithm engine, by a risk analysis microprocessor in communication with a tangible, non-transitory memory, a comprehensive risk value for a patient based upon incident data, transactional data and an estimated legal spend capacity, wherein the consumer transactional data comprises transaction amount, transaction time comprising a moment in time at which a transaction occurs, and wherein the probability value represents a risk associated with the lien is recovered, assigning, by the risk analysis microprocessor; selecting, by the risk analysis microprocessor and in response to the assigning, an appropriate risk factor relationship based upon the data and internal data; and storing, by the risk analysis microprocessor and in response to the selecting, the appropriate risk factor relationship in a database. The scoring of the profile can include risk factors and reward factors. The method can include an algorithm engine for executing the risk analysis, wherein the engine includes if-then logic that guides the automatic gathering of specific structured information from third party sources, stores that data, evaluates those combinations of data base of specific if then logic to guide the work flow and allow for automated document creation and work queuing to allow for the lien purchase transaction to occur and be perfected if appropriate or to be rejected and recommended for other steps as appropriate.

### DETAILED DESCRIPTION OF FIGURES

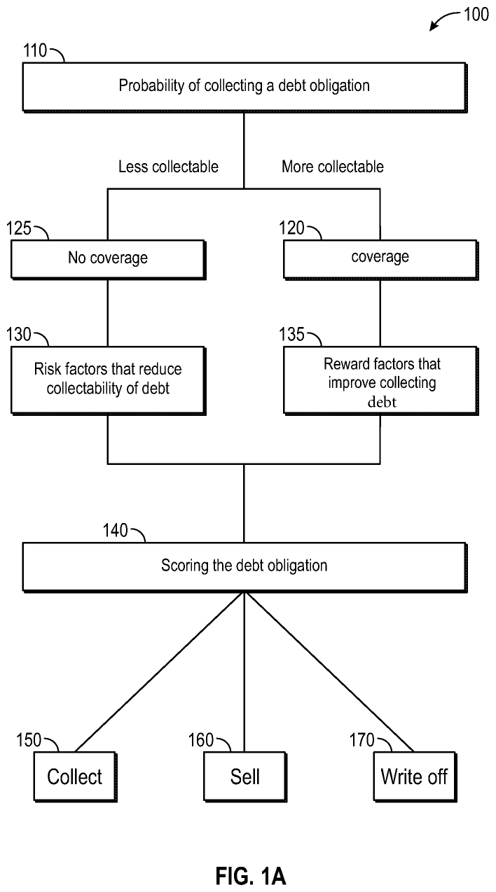

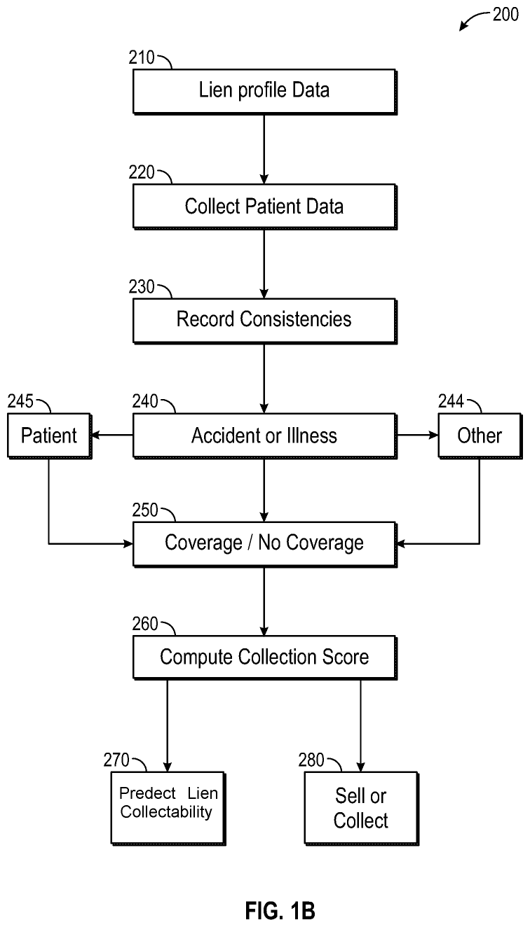

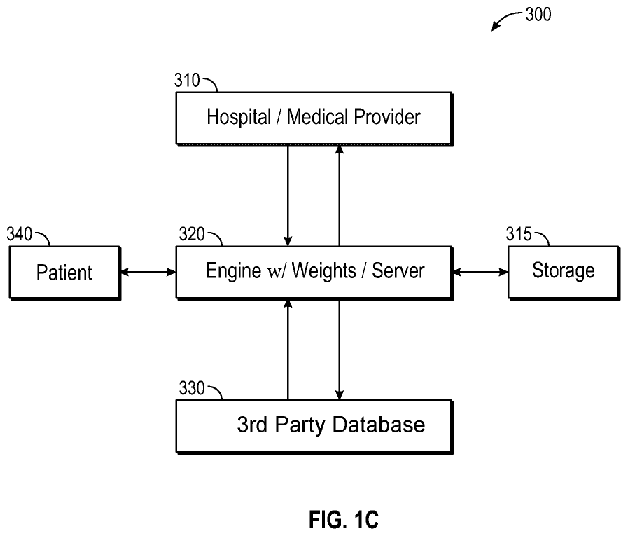

FIG. 1A is one embodiment of the system for assessing the collectability of a medical debt obligation;

FIG. 1B is another exemplary system for modeling the collectability of debt obligation;

FIG. 1C is an exemplary block diagram of example components of a computing device that may correspond to a server, in which one or more embodiments of the present disclosure may operate;

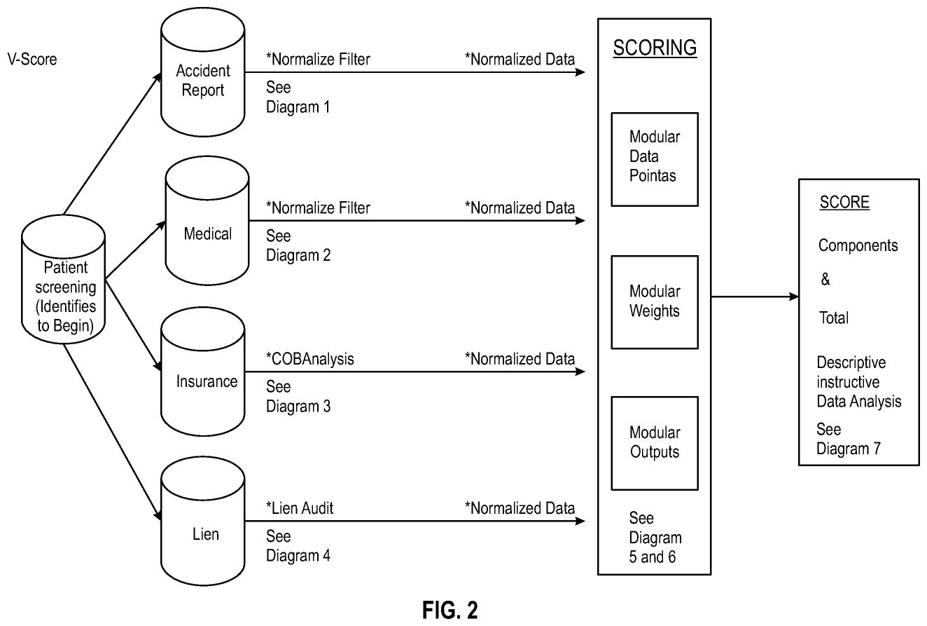

FIG. 2 is an exemplary block diagram of a system architecture for identifying debt obligations, according to an embodiment;

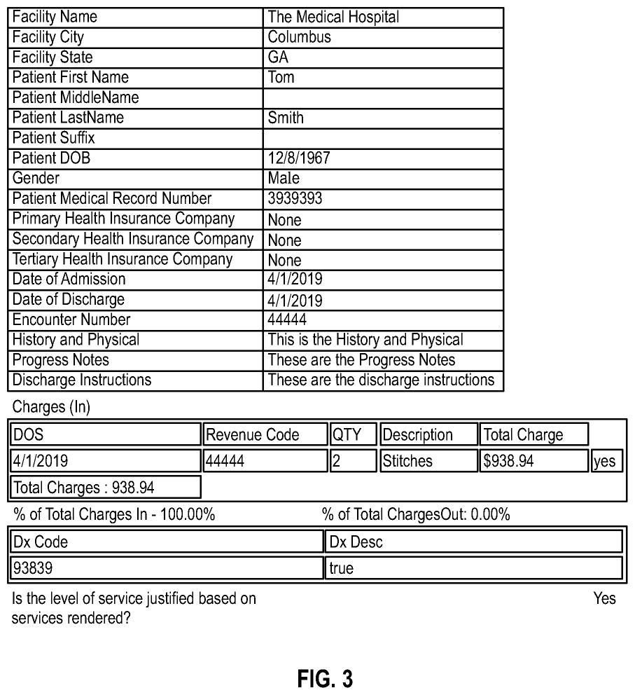

FIG. 3 exemplifies a data normalization process, e.g., for a medical report;

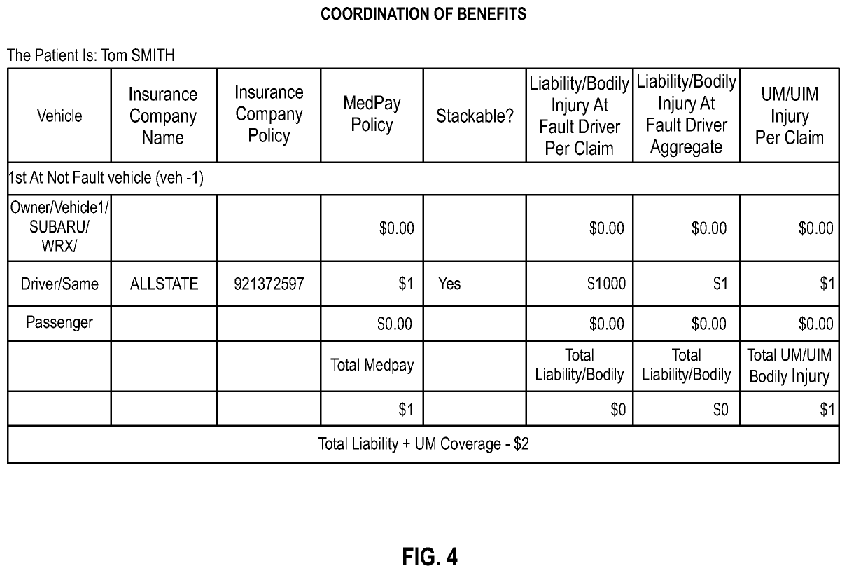

FIG. 4 exemplifies a data normalization process, e.g., for a coordination of benefits report;

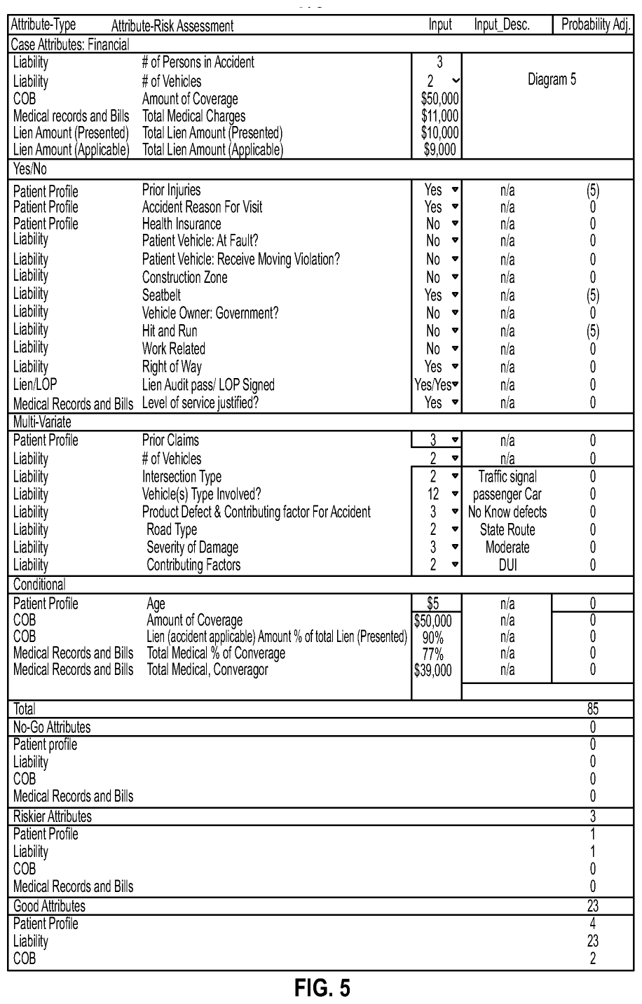

FIG. 5 exemplifies a data normalization process, e.g., for accident data and reports; and

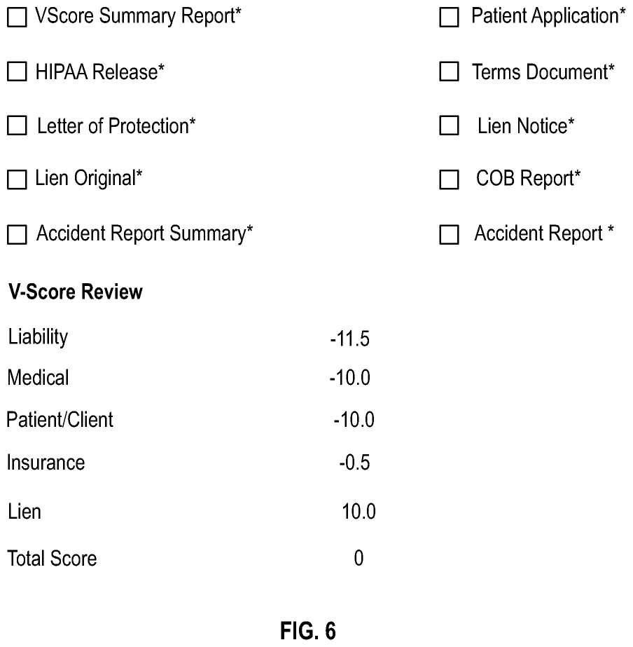

FIG. 6 illustrates how data points that can be used in scoring and can be modified, added or removed from the weighting and scoring process.

### DETAILED DESCRIPTION

The detailed description herein is presented for purposes of illustration only and not of limitation. For example, the steps recited in any of the method or process descriptions may be executed in any order and are not limited to the order presented. For the sake of brevity, conventional data networking, application development and other functional aspects of the systems (and components of the individual operating components of the systems) may not be described in detail herein. Although the present invention is described as relating to risk modeling of individual consumers, one of skill in the pertinent arts will recognize that the various embodiments of the invention can also apply to small businesses and organizations without departing from the spirit and scope of the present invention.

Specific embodiments relate to systems and methods for a risk-based scoring system to predict the collectability of a medical lien based on collection factors. This allows the medical lien or debt to risk assess, and ultimately collected or discharged by the health care provider.

In one embodiment, modeling risk includes obtaining accident/incident data, modeling and/or processing the health care data, and creating an output. The output may then be used to make business decisions. The variety of data (e.g., consumer data) in conjunction with several modeling/processing procedures to assess risk of recovering or not recovering a lien. Once the data is obtained by the evaluator, the rules engine can be programmed to create a set of questions structured to gather all of the required data for creating or modeling the data.

FIG. 1A shows exemplary aspects of the collectability of the medical lien or debt obligation modeled or assessed by the one embodiment of the invention. The method 100 includes an ultimate score that is the probability of that the medical lien or debt can be collected on an individual obligation scale. The score 140 is a measure of the likelihood that the medical lien or debt will be paid or collected by holder or medical provider. The score is independently assessed to each individual debt obligations and it varies based on collection risk factors, which may be known in the medial field. The score represents a more accurate indicator of ability to collect a specific or individual debt obligation. The score incorporates factors that improve the collectability of the debt and factors that reduce the collectability of the debt obligation. The amount of medical coverage (e.g., insurance) can be factored into the score, which allows an assessment of the collectability of the debt. The system allows health care providers with the ability to determine whether to pursue debt 150, write it off 170, or system sell it another 160.

The system can provide a computer implemented model for quantifying and assessing an the collectability of an individual debt obligation. The assessment can be based using risk factors 130, which reduce the probability that the debt may be collected; and reward factors 135, which increase the probability that a loan will be collectable. Optionally, a single element or factor (e.g., coverage 125 or no coverage 120) can be exceptionally weighted into the score. Thus, medical providers and others can assess or have an assessment of whether specific medical debt is collectable and act accordingly.

FIG. 1B shows another embodiment of the invention, which is more linear in nature. Specifically, this system 200 connects a specific medical lien or debt and creates a lien or debt profile 210. The system can collect further data, checks the consistency of the records 220, collects specific to cause of the treatment (accident or illness) 230, collects coverage data 250. This data is then used to compute the probability of or a score 260 for the collectability of the lien or debt. The system allows health care providers with the ability to predict collectability 270 or to determine whether to pursue debt, write it off, or system sell it to another 275.

FIG. 1C is an exemplary block diagram illustrating a system architecture 300 for identifying potential debt in which one or more embodiments of the present disclosure may operate through the use of an algorithm engine or scoring module 320 or service. In FIG. 1C, system architecture 300 includes an external database 330, storage 315, patient data (e.g., queried) 340, a scoring module 320 having rules and intelligence. The system may interact with third party databases 330 and the patient himself or herself 340. The rules may provide excessive weight to certain factors, which may be adjusted for specific situations. For example, medical record issues may be weighted more than income of the patient or treated person.

A debt obligation includes any obligation a consumer or patient has to pay a health care provider (e.g., a hospital). A hospital lien is a special right granted to hospitals and emergency services providers by Statute enabling them to receive payment from the first monies recovered from a negligent third-party by the injured victim. The phrase: “hospital lien” is actually short for “hospital and emergency services lien.” It is a right that attaches automatically and is often accompanied by written notice of a hospital lien, although this is not required. The lien applies only in emergency situations and to reasonable and necessary medical care provided as a result of the emergency for a set time period. A health care obligation is unsecured and is usually collectable from a related court judgement or payment. For convenience, a hospital debt is used herein to refer to a money or costs associated with providing healthcare to a patient for an injury from an incident or accident.

A lien holder is any person or entity that provides medical debt/lien collection services. A lien holder may deal in only in health care liens or obligations. A lien holder need not originate loans but may hold securities backed by debt obligations. A lender may be only a subunit or subdivision of a larger organization. A lien holder is any person or entity that is entitled to repayment of a medical payment loan. A properly recorded or legally sound lien may be given a larger weight in some instances.

Internal data is any data an evaluator possesses or acquires pertaining to a particular patient or circumstance. Internal data may be gathered before, during, or after a relationship between the health care provider and the consumer. Such data may include consumer demographic data. Consumer demographic data includes any data pertaining to a consumer. Consumer demographic data may include consumer name, address, telephone number, email address, employer and social security number. Consumer transactional data is any data pertaining to the particular transactions in which a consumer engages during any given time period.

The healthcare lien can be evaluated based on incident data, customer/patient information, hospital records, insurance or coverage information, billing integrity, and other information. The score will increase when there is data showing insurance coverage, accident fault attributable to another, medical records consistency, and coverage. If the patient is not covered by a commercial or government insurer, the health care person registering or pre-registering the registrar is further prompted to ask the patient questions presented on a multi-tiered questionnaire, stored in a database. During registration and/or pre-registration, the healthcare provider normally determines whether the patient's anticipated medical expenses will be covered by a third-party payer, such as an insurance company. If the patient is covered by a third-party payer, the patient portion of the anticipated medical expense is also determined and likelihood of collection on the lien is assessed by the rules.

In one example, the scoring for the liability component or risk aspect of the score can include different factors, these include but are not limited to, the number of claimants, the number of liable parties, the clear fault of the event itself, possible defenses of a potentially liable party, complicating legal factors such as whether government agencies will be involved, whether worker's compensation is involved, the weather, the type of roadway, the road conditions, aggravating factors such as DUI or reckless behavior. These can be weighted and evaluated and can provide an improved evaluation of the risk posed by this medical lien and issues that may be important for understanding the nature and quality of the underlying liability claim that the lien depends on.

The term “score” refers to a summation of scores assigned to relevant attributes within a category of interest.

Further, the final score can account for the future personal injury cause of action referred to in the lien, patient's proper identification with the lien, the proper statutory time limits, the filing requirements of the lien with the appropriate court/clerk office, the notice, and the billed amount. In other words, the score can account for whether the lien meets the legal requirements for a healthcare lien.

In one embodiment, the assessment can be essentially a pass/fail. If the medical bill is inaccurate, the medical bill may be sent back to the healthcare provider or hospital for correction. It is possible that if the hospital agrees to the recommendation of the adjusted balance and a new bill is issued and an adjustment to the lien has been filed that the purchase could go forward. There should be no purchases of a medical bill where the bill contains obvious errors unless the errors are of a nominal nature. Each element will be checked and negative elements will be noted and the ultimate score or assessment can be a decimal score, e.g., between 0-10. The factors and inquires may be weighted based on factors specific to a jurisdiction.

A composite score can be calculated based on a second formula that considers certain categories of negative scores that have been included. This can produce a final numeric score that will be compared to the recommended range of acceptable purchases.

In one embodiment, the method can include: calculating, by a risk analysis microprocessor in communication with a tangible, non-transitory memory, a comprehensive risk value for a patient based upon incident data, transactional data and an estimated legal spend capacity, wherein the consumer transactional data comprises transaction amount, transaction time comprising a moment in time at which a transaction occurs, and wherein the comprehensive consumer default risk value represents a risk associated with the lien is recovered, assigning, by the risk analysis microprocessor; selecting, by the risk analysis microprocessor and in response to the assigning, an appropriate risk factor relationship based upon the data and internal data; storing, by the risk analysis microprocessor and in response to the selecting, the appropriate risk factor relationship in a database; inserting, by the risk analysis microprocessor and in response to the storing, a data set annotation, wherein the data set annotation includes security information.

Scoring scoreable transactions against the predictive models may also produce account scores, i.e., scores assigned to accounts based on the scoreable transaction and/or the derived account-level pattern. By way of example, in account scoring, the pattern generated from the scoreable transaction is joined to model metadata using machine intelligence to generate an account-level score and reason codes. In one embodiment, the higher the score, the higher the probability that the account and/or account holder is at financial risk. As mentioned, scoring scoreable transactions against the predictive model may yield consolidated scores, i.e., scores assigned to a particular patients or debt obligations based on transactions across different accounts and/or even different account issuers. For example, the augmented scoreable transaction with its account-level scoring data may be joined to customer data to provide account holder-level detail. Using this information, the consolidated profile (e.g., the relational table containing the cumulative and smoothed variables used by the predictive models by account holder ID) may also be updated. Patient invoice-level patterns, account scoring and last account patterns may then be joined to the metadata using machine intelligence to generate an account holder-level score and reason codes. Still further, recently generated debt obligation-level and patient-level scores may also be combined to produce a single score per reporting period for each patient obligation according to specified parameters.

The present invention may also allow a lien buyer to create a risk model for use in targeting potential healthcare liens to acquire, make credit decisions regarding existing liens, and increase business with business health care partners.

In one embodiment, modeling risk includes obtaining accident/incident data, modeling and/or processing the health care data, and creating an output. The output may then be used to make business decisions. The variety of data (e.g., consumer data) in conjunction with several modeling/processing procedures to assess risk of recovering or not recovering a lien. Once the data is obtained by the evaluator, the rules engine can be programmed to create a set of questions structured to gather all of the required data for creating or modeling the data.

In another embodiment, once the claim has been purchased, the appropriate workflow can be guided intelligently by claim update information that is gathered during the claim monitoring phase of the case. This is essentially done through if/then logic. When the claim is resolved the appropriate workflow to resolve the can be guided by data gathered regarding settlement of the underlying personal injury claim.

In another embodiment the risk assessment of each item can be updated post purchase during the monitoring and collecting of the lien balance. As new data is gathered during this phase it may affect the risk score attributed to the outstanding balance/lien. If the claim becomes less likely for full recovery the score can be evaluated as riskier, if it becomes more likley for recovery it can be evaluated as less risky. This will help keep track of the current risk portfolio of all debt purchases at any given moment as opposed to their original assessment. This will help a portfolio manager understand the value and risk of the current portfolio. When the claim is resolved the appropriate workflow to resolve the can be guided by data gathered regarding settlement of the underlying personal injury claim.

FIG. 5 shows an example of risk model. In further embodiments, the population of current health care liens is subdivided into a plurality of further categories based on the amount of information available and the activity of such available data. In the example shown in (YOUR 3), the risk assessment can be based on information that is, e.g., a “0” or a “1) based on the response. Each character represents one month of available data, with the rightmost character representing the most current months and the leftmost character representing the earliest month for which data is available. For example, if the billing records are accurate and the insurance is adequate, then a score or FINAL composite score of “2” may be allocated to the lien. Based on the scale, one may decide whether to buy a healthcare provider lien.

Software instructions in the form of computer readable program code to perform embodiments of the invention may be stored, in whole or in part, temporarily or permanently, on a non-transitory computer readable medium such as a CD, DVD, storage device, diskette, tape, flash memory, physical memory, or any other computer readable storage medium. Specifically, the software instructions may correspond to computer readable program code that when executed by a processor(s), is configured to perform embodiments of the invention.

Further, one or more elements of the aforementioned computing system may be located at a remote location and connected to the other elements over a network. Further, embodiments of the invention may be implemented on a distributed system having a plurality of nodes, where each portion of the invention may be located on a different node within the distributed system. In one embodiment of the invention, the node corresponds to a distinct computing device. Alternatively, the node may correspond to a computer processor with associated physical memory. The node may alternatively correspond to a computer processor or micro-core of a computer processor with shared memory and/or resources.

### Examples

The first figure illustrates the overall scoring process for medical liens that are the result of a motor vehicle accident. Though this process data is collected, normalized/restructured, weighted based on modular processes, component scores, total scores, instructive indicators are provided to assist with later decision making. FIG. 2 shows an exemplary system or module illustrating the system according to specific embodiments.

FIG. 3 exemplifies the data normalization process for a motor vehicle accident report and report data. There are on average 2-3 major variations in report design per state. Therefore, there are somewhere between 100-120 different MVA report unique designs. Each of these designs are modular based on variations in the report details, such as the number of vehicles, the types of vehicles, the number of passengers involved in the accident, etc. Also, some law enforcement departments use slightly different layouts of the required design within their state. Each of the design variations can include differences in field labels, abbreviations used within data, etc. Also, each state will use slightly different wording on their data fields due to differences in statutory wording choices which will require that wording choice on the report mirror relevant wording choices for that state. For evaluation purposes however it is important to normalize this data as much as possible to allow for workflows to be created and while taking into account the important jurisdictional legal variations that exist create a somewhat standardized data structure that can apply to all 50 states and allow adequate risk analysis techniques to be applied. Collected data is placed into a uniform structure, where possible wording is normalized to a single standard, some state by state unique wording choices will be allowed to exist to allow for unique weighting measures to be applied based on unique jurisdiction based legal issues. This illustration shows a sample category within which data is normalized from the different kinds of MVA reports.

The scoring for the liability component score takes into account many different factors, these include but are not limited to, the number of claimants, the number of liable parties, the clear fault of the event itself, possible defenses of a potentially liable party, complicating legal factors such as whether government agencies will be involved, whether worker's compensation is involved, the weather, the type of roadway, the road conditions, aggravating factors such as DUI or reckless behavior. These are later weighted and evaluated as exemplified in FIGS. 5 and 6. This scoring will allow for a better understanding of the risk posed by this medical lien and issues that may be important for understanding the nature and quality of the underlying liability claim that the lien depends on.

FIG. 3 exemplifies the data normalization process for medical records and medical bills. This information is collected from the medical provider. This data can be structured and stored in unique ways. These may be based on legal jurisdictional requirements, medical record software design requirements, business decisions based on the provider, and/or variations in the way the data was collected from a third-party vendor. For evaluation purposes however it is important to normalize this data as much as possible to allow for workflows to be created and while taking into account the important jurisdictional legal variations that exist create a somewhat standardized data structure that can apply to states, various medical provider networks and types and allow adequate risk analysis techniques to be applied. Collected data is placed into a uniform structure, where possible wording or numbering is normalized to a single standard, some state by state unique wording choices will be allowed to exist to allow for unique weighting measures to be applied based on unique jurisdiction based legal issues. This illustration shows a sample output of normalized medical.

The scoring for the medical data takes into account the severity of the injuries, whether the injuries were related to the claim, whether the charges are appropriate and related to the claim, whether the gross charges fall within an acceptable range, and whether adequate data and documentation is present to allow it to be enforceable as a medical bill in this case. The weighting and scoring of this data occurs in FIGS. 4 and 5.

FIG. 6 exemplifies the data normalization and processing related to all available insurance coverages and policies as they apply to this MVA and related medical expenses. Data for insurance policies and coverages are collected based on data present in the MVA report. Once the policy data is collected it is normalized to a single data format. This is important because different insurers may use slight variations in their data structure compared to each other. Once that is completed, an automated coordination of benefits process is conducted taking into account the previously performed automated liability analysis based on the MVA report data. This aligns the coverage in the proper order as it applies to this MVA and performs necessary calculations to provide various necessary details regarding insurance coverage and carriers in this case. This is vital in setting up automated processes that will occur later in the scoring process and for subsequent tasks generally. This illustration shows a sample COB report structured based on normalized insurance policy data that has been collected.

The scoring of the insurance data measures whether there is adequate insurance related to this case. It takes into account but is not limited to the amount of charges, gross coverages that would applicable, the reliability of their carriers involved, the jurisdiction and venue involved, projected future care, projected litigation costs, and the number of potential claimants involved. The weighting and scoring of insurance category of data and related evaluations regarding the adequacy of coverage is performed in the moments illustrated by FIGS. 4 and 5.

FIG. 3 illustrates the normalization and examination of medical lien data that is collected. Each state may have slight variations in format and style and content regarding whag is required for perfecting medical liens. The liens and lien data are collected and compared to the jurisdictional standard so that normalized data points regarding the contents of each lien and quality of each lien can be applied to a universal standard. This is useful in understanding the adequacy of the lien, but also whether any inadequacies can be corrected and thereby what process changes in the future may improve the enforceability of future liens. The normalized lien data and evaluative data created here are necessary to allow for tasks and processes that will occur later including scoring.

The weighting and scoring of the lien include but is not limited to the following: the timing of the filing, whether the lien form contains complete and accurate data, whether the lien was filed in the proper court, and whether the proper notices have been sent informing other parties of the filing. The weighting and the scoring of the lien data is performed in FIGS. 4 and 5.

FIG. 4 illustrates how data points that will be used in scoring can be modified, added or removed from the weighting and scoring process. Because the data has been normalized it is possible to apply somewhat standard scoring techniques This is an interface that applies to all of the normalized data that has been processed in a case so far. This process can apply to any data point present within the platform. The modular nature of this interface can be adaptable state by state but still allows for a certain level of uniformity as well. Also in this interface the reflected values based on the normalized data present can be seen. Underlying modular algorithms upon which these weights are based are visible in FIGS. The purpose is to reach a significant numeric score that is representative of the quality of risk of each category of data and be able to articulate specific issues that are instructive as to the reason for the score and on how the case should proceed.

FIG. 6 is an example of an interface that illustrates to component scores and the total score which represents the risk assessment of the medical lien. Not indicated in this figure are additional instructive factors but a brief list of factors is available as a result of the process in addition to the numeric score.

While the invention has been described with respect to a limited number of embodiments, those skilled in the art, having the benefit of this disclosure, will appreciate that other embodiments can be devised which do not depart from the scope of the invention as disclosed herein. Accordingly, the scope of the invention should be limited only by the attached claims.

1. A method for assessing debt obligations to a health care provider on an individual basis, comprising:
a. obtaining over a communication network connection, metadata corresponding to one or more potential health care debts from sources to obtain repayment information about each of the one or more debts; and
b. transforming the data into a profile for each debt which corresponds to an individual's obligation;
c. scoring the profile for predicted repayment of the debts, wherein the score correlates with the collectability of the debt.

2. The method according to claim 1, wherein the score is a summation of the weight score value assigned to each matched attribute.

3. The method according to claim 1, wherein each of the plurality of categories of interests is selected from a group consisting of lien information, medical coverage, and cause of medical services.

4. The method according to claim 1, wherein the scoring of the profile is risk factors and reward factors.

5. The method according to claim 4, wherein the risk factors are assigned a risk factor weight and the reward factors are assigned a reward factor weight.

6. The method according to claim 4, wherein the risk factors are subcategorized and sub-weighted.

7. The method according to claim 4, wherein third-party data is retrieved to weight the risk factors or the reward factors.

8. The method according to claim 4, wherein the risk factors and the reward factors are given a numerical score.

9. A method comprising:
Obtaining over a communication network connection, metadata corresponding to one or more potential health care debts from sources to obtain repayment information about each of the one or more debts; and
Scoring with an algorithm engine, by a risk analysis microprocessor in communication with a tangible, non-transitory memory, a comprehensive risk value for a patient based upon incident data, transactional data and an estimated legal spend capacity, wherein the consumer transactional data comprises transaction amount, transaction time comprising a moment in time at which a transaction occurs, and wherein the probability value represents a risk associated with the lien is recovered, assigning, by the risk analysis microprocessor;
selecting, by the risk analysis microprocessor and in response to the assigning, an appropriate risk factor relationship based upon the data and internal data; and
storing, by the risk analysis microprocessor and in response to the selecting, the appropriate risk factor relationship in a database.

10. The method according to claim 1, wherein the scoring of the profile is risk factors and reward factors.

11. A method of claim 9, further comprising an algorithm engine for executing the risk analysis, wherein the engine includes if-then logic that guides the automatic gathering of specific structured information from third party sources, stores that data, evaluates those combinations of data base of specific if then logic to guide the work flow and allow for automated document creation and work queuing to allow for the lien purchase transaction to occur and be perfected if appropriate or to be rejected and recommended for other steps as appropriate.

12. A method of claim 9, further comprising if-then logic that guides the automatic gathering of specific structured information from third party sources, stores that data, evaluates those combinations of database of specific if then logic to guide the work flow and allow for automated document creation and work queuing to allow for the monitoring of the purchased liens.

13. A method of claim 9, further comprising if-then logic that guides the automatic gathering of specific structured information from third party sources, stores that data, evaluates those combinations of data base of specific if then logic to allow for the update of the risk score of a lien post purchase to allow for the ongoing assessment of the present time risk assessment of a debt item and debt portfolio to be known.

14. A method of claim 9, further comprising if-then logic that guides the automatic gathering of specific structured information from third party sources, stores that data, evaluates those combinations of data base of specific if then logic to guide the work flow and allow for automated document creation and work queuing to allow for the collection of payment of the lien and the appropriate filings and documentation processing to occur to complete lien payment transaction appropriately.

15. The method of claim 1, further comprising transmitting the score to a third party different from provider if said score reflects a low level of financial risk.

16. The method of claim 1, further comprising transmitting the score to a third party different from provider if said score reflects a high level of financial risk.
